(19) United States
(12) Patent Application Publication
(10) Pub. No.: US 2025/0000605 A1
(43) Pub. Date: Jan. 2, 2025

(54) IMPLANT PLACEMENT GUIDES AND METHODS

(71) Applicants: Icahn School of Medicine at Mount Sinai, New York, NY (US); Monogram Orthopaedics Inc., Austin, TX (US)

(72) Inventors: Kamran SHAMAEI, Austin, TX (US); Douglas B. UNIS, Brooklyn, NY (US)

(21) Appl. No.: 18/807,524

(22) Filed: Aug. 16, 2024

Related U.S. Application Data
(63) Continuation of application No. PCT/US2023/062713, filed on Feb. 16, 2023.
(60) Provisional application No. 63/268,070, filed on Feb. 16, 2022.

(51) Int. Cl. A61B 90/00; A61B 34/20

(52) U.S. Cl. CPC A61B 90/37; A61B 2034/2055; A61B 2090/365

(57) ABSTRACT

Facilitating proper object placement during a surgical procedure includes tracking position and location of a physical object as the object is moved in a surgical environment to position and place the object in a desired position/placement relative to patient anatomy, presenting augmented reality (AR) element(s) on a display device such that the AR element(s) overlay a view of the surgical environment to aid in the proper positioning and placement of the object relative to the patient anatomy, and visually indicating on the display device that/when the object has been moved into the desired position and placement.

### BACKGROUND

An objective of reconstructive surgery is to precisely place implant(s) on a patient's bone that has been cut to interface with such implant(s). Elaborate instrumentation, including navigation and robotics, make suitable bone cuts.

### SUMMARY

Shortcomings of the prior art are overcome and additional advantages are provided through the provision of, in a first embodiment, a computer-implemented method. The method incudes determining a desired position and placement for a physical object to be positioned and placed relative to a patient anatomy, the desired position and placement being dictated by a surgical plan for a patient; presenting, on a display device, an augmented reality (AR) element overlying a view of a surgical environment, wherein the surgical environment includes the patient anatomy, and wherein the presenting positions and places the AR element at the desired position and placement for the physical object to facilitate proper placement of the physical object relative to the patient anatomy; tracking position and location of the physical object as the physical object is moved in the surgical environment to position and place the physical object relative to the patient anatomy; and based on the position and location of the physical object matching to the desired position and placement for the physical object, visually indicating on the display device that the physical object has been moved into the desired position and placement relative to the patient anatomy.

In embodiments, the method can optionally further include obtaining a video stream depicting the view of the surgical environment, including a depiction of the patient anatomy; and presenting the video stream on the display device, wherein the presenting the AR element on the display device augments the video stream to include the AR element at the desired position and placement for the physical object to be placed relative to the patient anatomy as depicted in the video stream.

In embodiments, the method can optionally further include, as part of presenting the video stream, and as the physical object is positioned relative to the patient anatomy, zooming-in on an area of the surgical environment where the AR element is presented.

In embodiments, the display device optionally includes a transparent display through which a user sees the view of the surgical environment, wherein the AR element is presented on the transparent display and augments the user's view of the surgical environment through the transparent display.

In embodiments, the transparent display is optionally provided as part of a smart wearable glasses device.

In embodiments, the visually indicating optionally comprises modifying a visual presentation of the AR element on the display device.

In embodiments, the AR element is optionally initially at least partially a first color or pattern and wherein the visually indicating comprising changing the first color or pattern to a different color or pattern.

In embodiments, the tracking the position and location of the physical object is optionally performed using at least one of: one or more markers on the physical object; a shape matching algorithm that identifies position and location of the physical object in the view based on a known shape of the physical object; or tracking location of at least one other physical object that has a fixed, known location relative to the physical object.

In embodiments, the method can optionally further include tracking location and position of the patient anatomy in the surgical environment, wherein, based on the patient anatomy being repositioned, the presenting the AR element repositions the AR element to maintain the AR element at the desired position and placement for the physical object to be placed relative to the patient anatomy.

In embodiments, the surgical plan optionally comprises one or more desired cuts to patient anatomy, wherein the method determines that the one or more desired cuts have been made to the patient anatomy and further performs the determining the desired position and placement for the physical object and the presenting the AR element based on the one or more desired cuts having been made.

In embodiments, the AR element is optionally a first AR element and wherein the method further comprises presenting, on the display device, a second AR element that overlays the physical object in the view of the surgical environment.

In embodiments, the physical object is optionally an implant.

In embodiments, the AR element is optionally a digital model of the physical object.

In alternative embodiments, a computer system is provided that includes a memory; and a processor in communication with the memory, where the computer system is configured to perform any of the foregoing methods. In other alternative embodiments, a computer program product is provided that includes a computer readable storage medium readable by a processing circuit and storing instructions for execution by the processing circuit for performing any of the foregoing methods.

Additionally or alternatively, shortcomings of the prior art are overcome and additional advantages are provided through the provision of, in a second embodiment, a computer-implemented method. The method incudes tracking position and location of a physical object in a surgical environment as the physical object is moved in the surgical environment; presenting, on a display device, an augmented reality (AR) element that overlays the physical object in a view of the surgical environment, the view showing the physical object and a patient anatomy relative to which the physical object is to be positioned and placed in accordance with a desired position and placement for the physical object as dictated by a surgical plan for a patient, wherein the presenting maintains, based on the tracking, the AR element overlaying the physical object in the view as the physical object is moved in the surgical environment; and based on the position and location of the physical object matching to the desired position and placement for the physical object, visually indicating on the display device that the physical object has been moved into the desired position and placement relative to the patient anatomy.

In embodiments, the method optionally further includes obtaining a video stream depicting the view of the surgical environment, including a depiction of the patient anatomy; and presenting the video stream on the display device, wherein the presenting the AR element on the display device augments the video stream to include the AR element overlaying the physical object in the view of the surgical environment.

In embodiments, the method optionally further includes, as part of presenting the video stream, and as the physical object is positioned relative to the patient anatomy, zooming-in on an area of the surgical environment showing a position of the desired position and placement for the physical object.

In embodiments, the display device optionally comprises a transparent display through which a user sees the view of the surgical environment, wherein the AR element is presented on the transparent display and augments the user's view of the surgical environment through the transparent display.

In embodiments, the transparent display is optionally provided as part of a smart wearable glasses device.

In embodiments, the visually indicating optionally comprises modifying a visual presentation of the AR element on the display device.

In embodiments, the AR element is optionally initially at least partially a first color or pattern and wherein the visually indicating comprising changing the first color or pattern to a different color or pattern.

In embodiments, the tracking the position and location of the physical object is optionally performed using at least one of: one or more markers on the physical object; a shape matching algorithm that identifies position and location of the physical object in the view based on a known shape of the physical object; or tracking location of at least one other physical object that has a fixed, known location relative to the physical object.

In embodiments, the AR element is optionally a first AR element and wherein the method further comprises presenting, on the display device, a second AR element positioned and placed relative to the patient anatomy to indicate the desired position and placement for the physical object.

In embodiments, the AR element is optionally a digital model of the physical object.

In embodiments, the physical object is optionally an implant.

In alternative embodiments, a computer system is provided that includes a memory; and a processor in communication with the memory, where the computer system is configured to perform any of the foregoing methods. In other alternative embodiments, a computer program product is provided that includes a computer readable storage medium readable by a processing circuit and storing instructions for execution by the processing circuit for performing any of the foregoing methods.

Additional features and advantages are realized through the concepts described herein.

### BRIEF DESCRIPTION OF THE DRAWINGS

Aspects described herein are particularly pointed out and may be distinctly claimed, and objects, features, and advantages of the disclosure are apparent from the detailed description herein taken in conjunction with the accompanying drawings in which:

FIGS. 1A-1H and 2 depict example environments to use or incorporate aspects described herein;

FIG. 3 depicts one example of a computer system and associated devices to incorporate and/or use aspects described herein;

FIG. 4 depicts one example of a smart eyewear device; and

FIGS. 5 and 6 depict example processes for tracking, locating, and/or placing an object relative to patient anatomy during a surgical procedure, in accordance with aspects described herein.

### DETAILED DESCRIPTION

Despite efforts to make accurate bone cuts, the placement of implants onto the bone, once the surgeon and/or robot makes the cuts, remains highly subjective and subject to surgeon skill. By way of non-limiting example, a surgeon may use a guidance application associated with a navigated surgical robot to virtually place implants and execute the related cuts based on a defined surgical plan. Once the surgeon finishes the cuts, however, there is no method to ensure the accuracy of implant placement relative to the planned placement—the process of placing the implants on the bone is subject to surgeon skill and is imprecise. One problem is that surgeons currently have no way of knowing whether they have placed the implant in the intended and planned position dictated by the surgical plan. While significant efforts are made to make accurate cuts, currently the surgeon manually places the implant(s) on those cuts with little to no guidance.

Aspects described herein propose novel methods of assisting a surgeon and/or robotic instrument for more accurate implant placement, for instance placement closer and more consistent with the planned placement of the implant, during a surgical procedure.

In a first example embodiment, a method proposes the use of augmented overlay(s) (e.g. augmented reality (AR) overlay) to display on/as part of an RGB stream (e.g. video stream) of the surgical scene the location and position that the implant is to be placed. Such an AR overlay could present a rendered implant volume in a varying and selectable level of opacity (e.g. solid or semi-transparent). The implant representation can be projected onto the bone in the position dictated by the planned virtual placement. This may be akin to a registration overlay in which the surgeon lines-up a bone model overlay with the actual bone, except that the surgeon, possibly with the assistance of software, is to line-up the implant to be placed with the target displayed as an AR overlay on top of the cut bone.

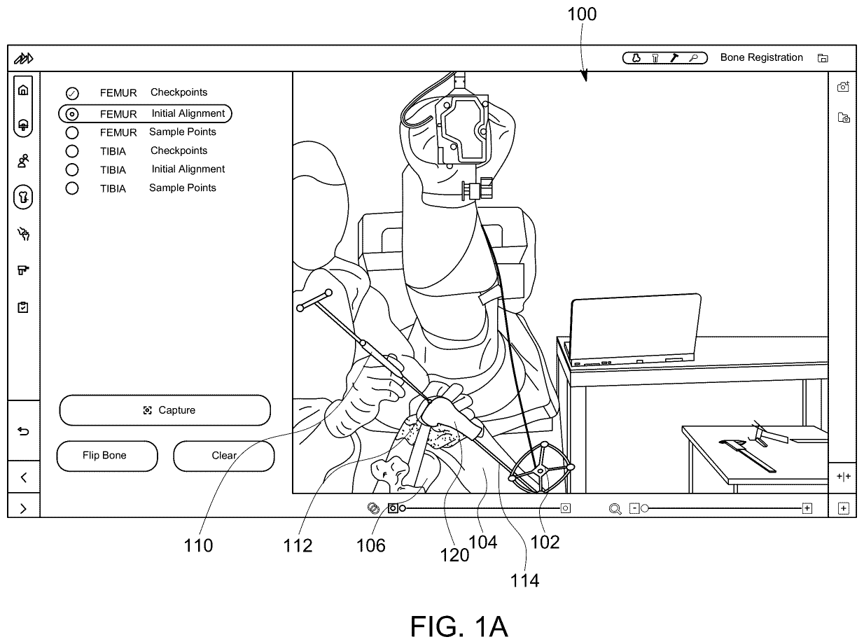

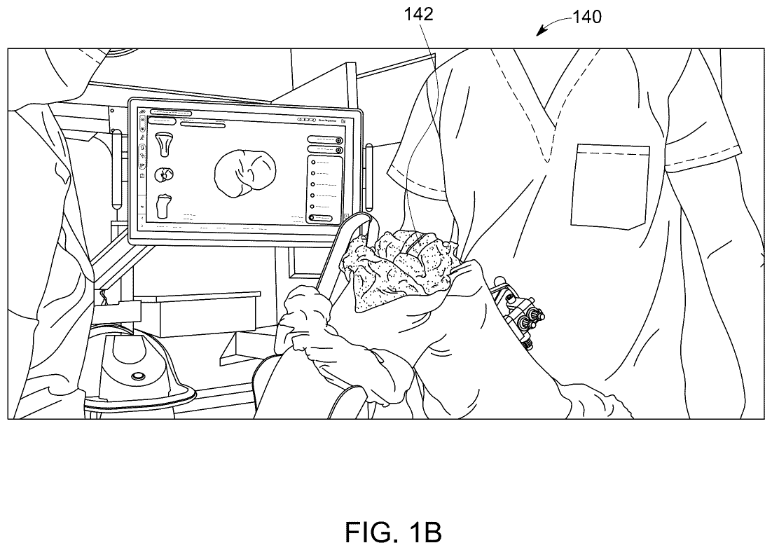

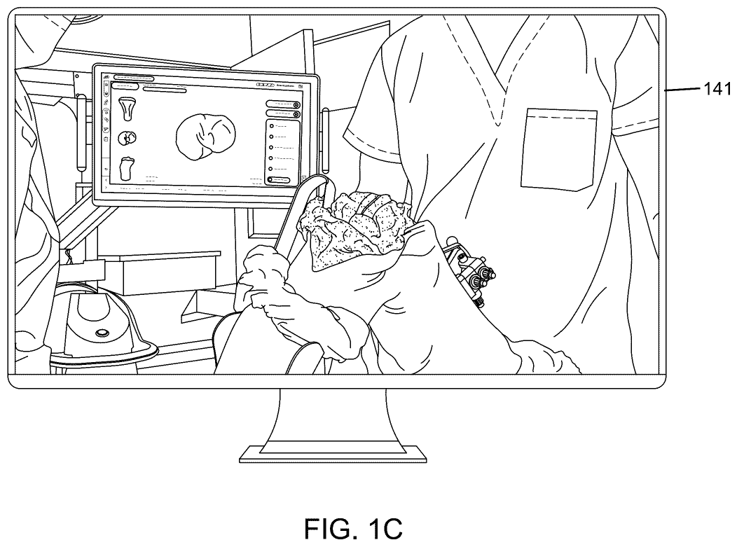

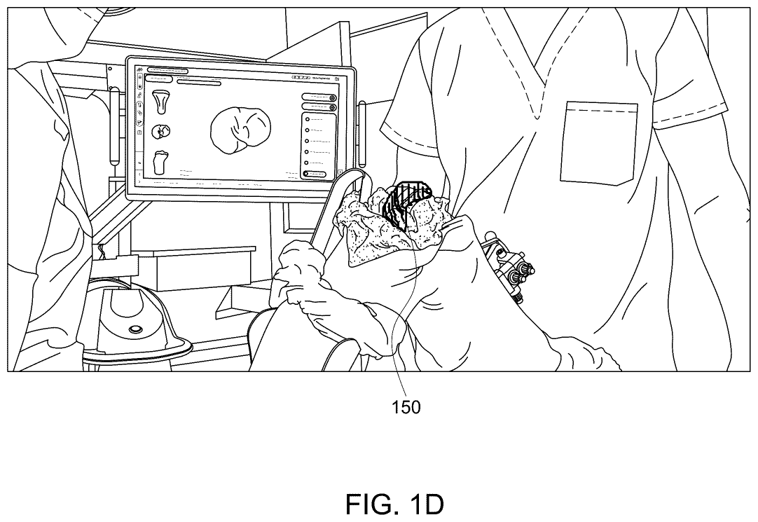

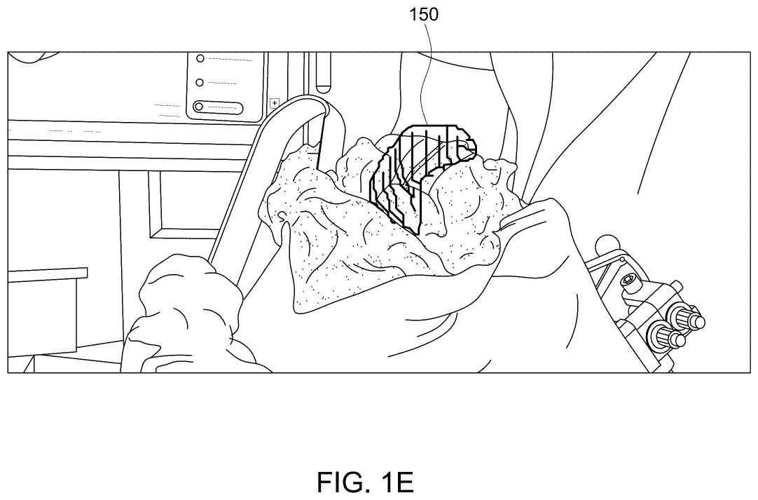

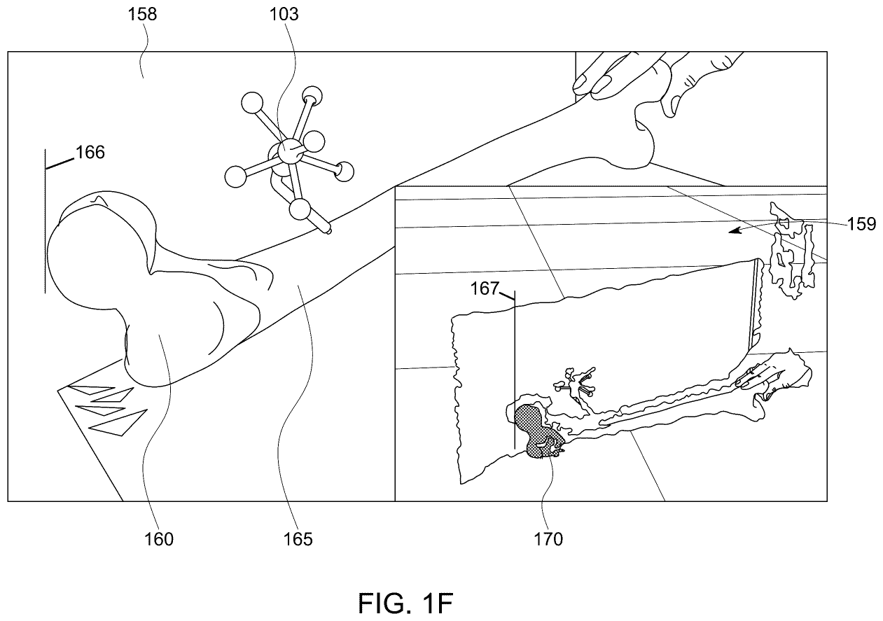

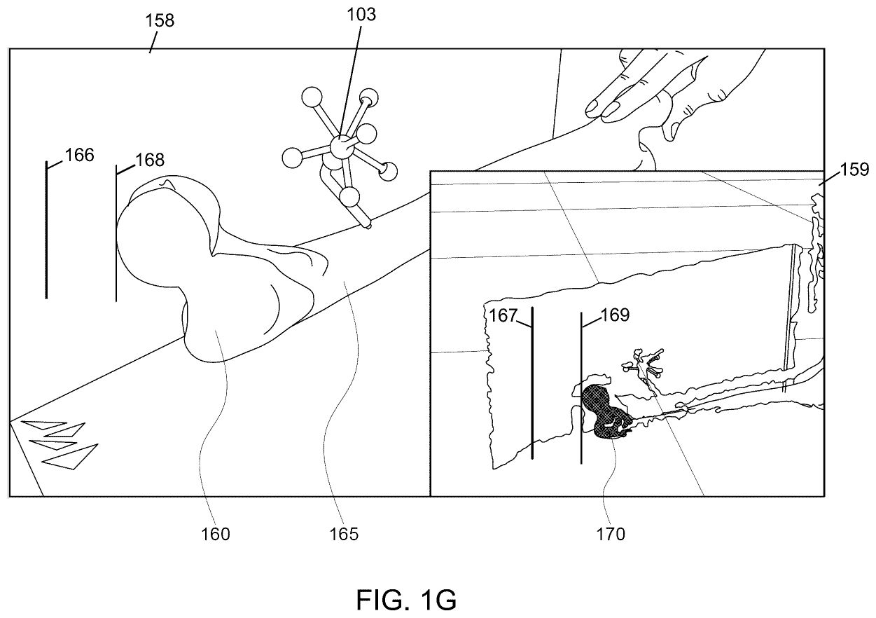

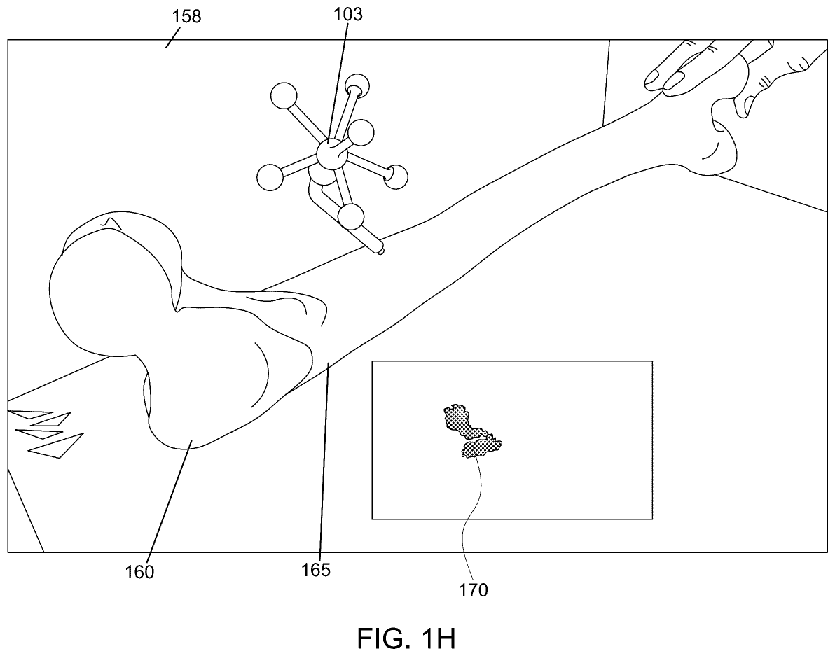

Referring to FIG. 1A, 100 shows an example stream/view of a surgical environment in which a surgery is in-progress. The view may be provided by a camera feed/stream, and a computer system can overlay/superimpose AR element(s) over the view and display the view with AR element(s) (i.e. “augmented view”) on a display screen. Additionally or alternatively, a user (e.g. a surgeon) could wear smart glasses or other wearable device(s) and view the environment through a transparent display(s) (such as transparent lenses with active displays built therein), and the AR element(s) could be presented on the transparent display to provide the augmented view for the user.

The view 100 depicts a fixed tracking array 102 and upper and lower leg portions, 104 and 106, respectively, of a patient. A registration probe 110, which might be used in a registration process to identify exact position(s) of objects, has a probe tip 112 positioned at the end of the femur 106 in this example. FIG. 1A also depicts a guide line 114 (presented in a given color, such as white) as a central axis line representing an axis of the probe 110, extending from the probe 110 (from the probe tip 112 in this example) toward the end of the femur. This guide line 114 may be presented for the surgeon as an augmented reality (AR) element and can help the surgeon properly orient the probe, for instance such that this line extends as close as possible through the central axis of the patient bone to point the line at the patient's hip center. This can be utilized to facilitate coaxial alignment between the probe and actual patient bone.

Shown also in FIG. 1A is an implant model 120 presented as an AR element imposed in the view 100 of the environment, and particularly at a location, e.g. adjacent/onto the patient bone, in a position dictated by the planned virtual placement of the implant. The implant model 120 can be repositioned in the event that the patient anatomy is repositioned, on account that the location of fixed objects, such as the patient bone, in space is known based on position of the tracking array 102.

FIG. 1B depicts another example view 140 of what is visible to a surgeon. This view can be presented on a display (as in FIG. 1C on display 141). Position of the bone 142 may be tracked using rigid markers/array (not pictured) attached to the bone, so a computer system receiving/processing the tracking data is aware of exactly where the bone is in space. Furthermore, the computer system knows what cuts to the bone have been made and exactly where the cuts are located, for instance because they may be executed robotically. Referring to FIG. 1D, an AR overlay 150 is added to the view on the display exactly where the implant is to be positioned and the surgeon can view the display to identify this position. Ultra-high definition camera(s) (e.g. 8K or greater resolution) can be utilized so that the surgeon can zoom-in on the view, if desired, as shown in FIG. 1E, to more clearly and exactly identify the intended position of the implant. The surgeon can zoom-in to view the AR overlay 150 while the surgeon positions the physical implant (which may be viewable in the view as well) in order to match the position of the implant to the position of the AR overlay (which is taken to be the precise and proper location for the implant).

In an additional or alternative example embodiment, the position of the physical implant in space is tracked. The AR model of the implant, represented as an overlay to the implant in the view, may be presented in a first color and/or pattern in the view until the implant is in the proper position adjacent/on the patient bone, at which time the AR overlay of the implant may be changed to a different color and/or pattern to indicate proper placement. More generally, visual indicator(s) associated with the AR model of the implant could indicate when the actual, physical implant has been moved into proper position relative to the patient anatomy, in comparison to when the implant is not in the proper position. Implant position and location could be indicated by an AR overlay, and implant position and location could be determined using any desired approach. In one approach, removable markers can be affixed to the implant to make it trackable. Additionally or alternatively, shape matching algorithms can be used to identify the implant in the scene and track it (a benefit of an RGB camera).

In this latter regard, and referring now to FIGS. 1F-1H, depicted is an example of object-shape-based tracking. An environment 158 contains patient anatomy 165 (e.g. a bone) with an object (for example an implant) 160. Here the object 160 is affixed to the bone 165 but tracking of the object can occur regardless whether the object 160 is actually affixed to the patient anatomy. Markerless tracking can be provided, in which aspects can track (e.g. based on a stream from RGB camera(s)) the location of object 160 in space based on its shape. The object 160 can also be drawn as an AR overlay 170 (in stipple in this example, though this could be filled with color(s) to complete the shape and periphery thereof) on a display showing a virtual depiction of the environment, as shown by view 159.

The exact location of objects in view can be known based at least in part on a known location of the fixed tracking array 103 and/or, in the case of object 160, by using shape-matching algorithm(s) to track the object by way of its shape, which is expected to be unique and discernible from other shapes in the environment. The vertical lines 166 and 167 in FIG. 1F (which could be presented in a first color, such as yellow) are presented for convenience to indicate, respectively, a first position of the object 160 in environment 158 and corresponding first position of the AR model 170 in view 159. FIG. 1G presents a view after the object 160 has been shifted/moved into a different position (in this example on account that the bone 165 to which the object 160 is affixed has moved). The vertical lines 168 and 169 in FIG. 1G are presented for convenience to indicate, respectively, a second position of the object 160 in environment 158 and corresponding second position of the AR model 170 in view 159. Vertical lines 168 and 169 after the movement can be presented in a second color, different from the first color, for instance in green. The AR model 170 can therefore be used to reflect updated positioning and therefore movement of the object 160 in real space, as a virtual overlay. In a particular embodiment of implant placement, the implant can be placed on the bone and positioned, while being tracked based on its shape and presented as AR element(s) on a display (e.g. the dots/stipple in FIG. 1F-1H). When the implant is in the desired location, which may be programmatically/computationally determined for instance, based on the exact virtual placement of the implant in surgical planning, a color or other visual indication of or associated with the AR overlay can be changed from one color (or indication) to another to indicate to the surgeon that the implant is properly position and placed.

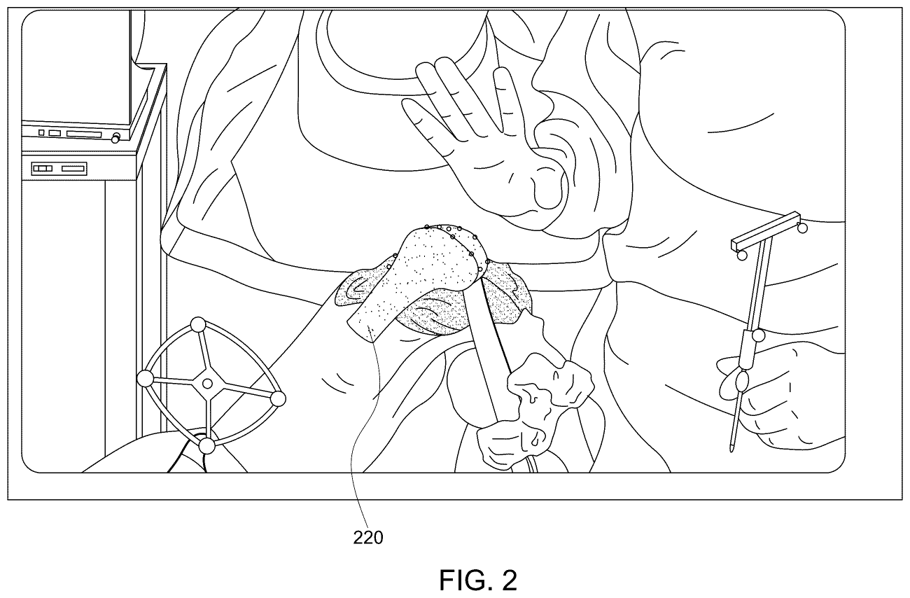

FIG. 2 depicts an example in which the AR overlay 220 has changed to a different color (such as a bright yellow) to indicate to a viewer that the surgeon has placed the implant (not visible here, as it is covered by the AR overlay 220) in the proper position.

In yet another example embodiment, instrument(s) that hold the implant could be tracked—for example, retroreflective markers could be affixed to the instruments for IR tracking of the instruments(s) and therefore also the implant, since the position of the implant relative to the instrument(s) remains fixed as the implant is moved into position.

Aspects provide improved accuracy and a better user experience—the surgeon no longer has to rely solely on use his or her judgment to place the implant(s) on cut bone, and he/she can position the implants exactly where they are supposed to go based on their surgical plan. This may be helpful for any navigated or robotic surgical procedure where implants need to be placed based on a surgical plan. It is noted that examples discussed herein related to implant placement on/against bone, though aspects described herein apply more generally to placement of objects (for instance implants) on/against/adjacent any patient anatomy.

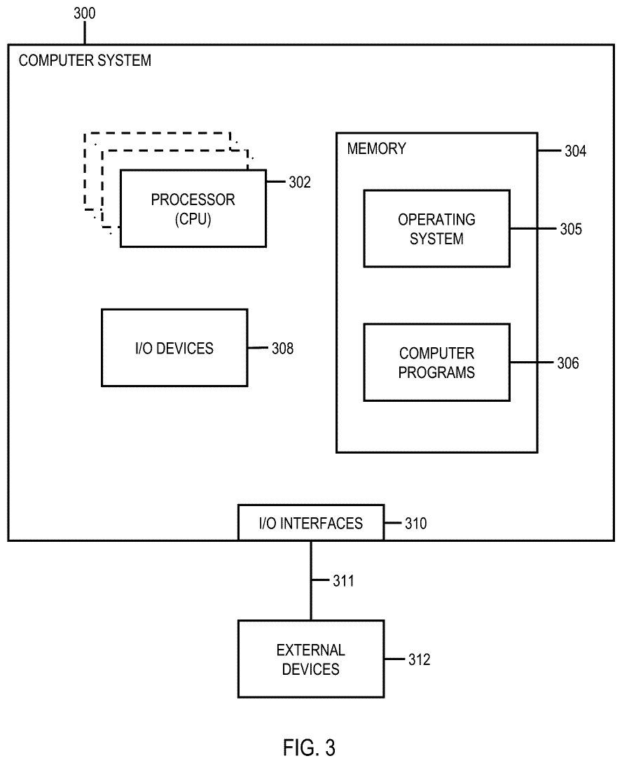

Processes described herein may be performed singly or collectively by one or more computer systems, such as one or more systems that are, or are in communication with, a camera system, tracking system, and/or AR system, as examples. FIG. 3 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 3 shows a computer system 300 in communication with external device(s) 312. Computer system 300 includes one or more processor(s) 302, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 302 can also include register(s) to be used by one or more of the functional components. Computer system 300 also includes memory 304, input/output (I/O) devices 303, and I/O interfaces 310, which may be coupled to processor(s) 302 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 304 can be or include main or system memory (e.g., Random Access Memory) used in the execution of program instructions, storage device(s) such as hard drive(s), flash media, or optical media as examples, and/or cache memory, as examples. Memory 304 can include, for instance, a cache, such as a shared cache, which may be coupled to local caches (examples include L1 cache, L2 cache, etc.) of processor(s) 302. Additionally, memory 304 may be or include at least one computer program product having a set (e.g., at least one) of program modules, instructions, code or the like that is/are configured to carry out functions of embodiments described herein when executed by one or more processors.

Memory 304 can store an operating system 305 and other computer programs 306, such as one or more computer programs/applications that execute to perform aspects described herein. Specifically, programs/applications can include computer readable program instructions that may be configured to carry out functions of embodiments of aspects described herein.

Examples of I/O devices 308 include but are not limited to microphones, speakers, Global Positioning System (GPS) devices, RGB and/or IR cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (312) coupled to the computer system through one or more I/O interfaces 310.

Computer system 300 may communicate with one or more external devices 312 via one or more I/O interfaces 310. Example external devices include a keyboard, a pointing device, a display, and/or any other devices that enable a user to interact with computer system 300. Other example external devices include any device that enables computer system 300 to communicate with one or more other computing systems or peripheral devices such as a printer. A network interface/adapter is an example I/O interface that enables computer system 300 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 310 and external devices 312 can occur across wired and/or wireless communications link(s) 311, such as Ethernet-based wired or wireless connections. Example wireless connections include cellular, Wi-Fi, Bluetooth®, proximity-based, near-field, or other types of wireless connections. More generally, communications link(s) 311 may be any appropriate wireless and/or wired communication link(s) for communicating data.

Particular external device(s) 312 may include one or more data storage devices, which may store one or more programs, one or more computer readable program instructions, and/or data, etc. Computer system 300 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 300 may be operational with numerous other general purpose or special purpose computing system environments or configurations. Computer system 300 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

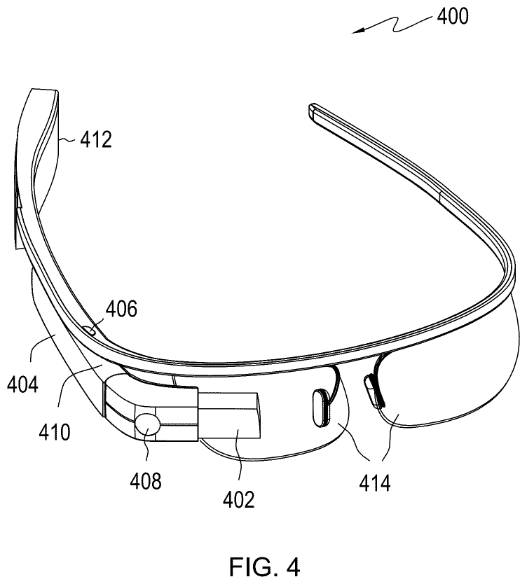

FIG. 4 depicts another example of a computer system to incorporate and use aspects described herein. FIG. 4 depicts an example eyewear based wearable device, for instance a wearable smart glasses device to facilitate presentation of AR elements to a wearer of the device. Device 400 can include many of the same types of components included in computer system 300 described above. In the example of FIG. 4, device 400 is configured to be wearable on the head of the device user. The device includes a display 402 that is positioned in a peripheral vision line of sight of the user when the device is in operative position on the user's head. Suitable displays can utilize LCD, CRT, or OLED display technologies, as examples. Lenses 414 may optionally include active translucent displays, in which an inner and/or outer surface of the lenses are capable of displaying images and other content. This provides the ability to impose this content directly into the line of sight of the user, overlaying at least part of the user's view to the environment through the lenses. In particular embodiments described herein, content presented on the lens displays are AR elements overlaying a stream from camera(s) depicting a surgical environment/theater.

Device 400 also includes touch input portion 404 that enable users to input touch-gestures in order to control functions of the device. Such gestures can be interpreted as commands, for instance a command to take a picture, or a command to launch a particular service. Device 400 also includes button 406 in order to control function(s) of the device. Example functions include locking, shutting down, or placing the device into a standby or sleep mode.

Various other input devices are provided, such as camera 408, which can be used to capture images or video. The camera can be used by the device to obtain image(s)/video of a view of the wearer's environment to use in, for instance, capturing images/videos of a scene. Additionally, camera(s) may be used to track the user's direction of eyesight and ascertain where the user is looking, and track the user's other eye activity, such as blinking or movement.

One or more microphones, proximity sensors, light sensors, accelerometers, speakers, GPS devices, and/or other input devices (not labeled) may be additionally provided, for instance within housing 410. Housing 410 can also include other electronic components, such as electronic circuitry, including processor(s), memory, and/or communications devices, such as cellular, short-range wireless (e.g., Bluetooth), or Wi-Fi circuitry for connection to remote devices. Housing 410 can further include a power source, such as a battery to power components of device 400. Additionally or alternatively, any such circuitry or battery can be included in enlarged end 412, which may be enlarged to accommodate such components. Enlarged end 412, or any other portion of device 400, can also include physical port(s) (not pictured) used to connect device 400 to a power source (to recharge a battery) and/or any other external device, such as a computer. Such physical ports can be of any standardized or proprietary type, such as Universal Serial Bus (USB).

Aspects of the present invention may be a system, a method, and/or a computer program product, any of which may be configured to perform or facilitate aspects described herein. Computer system configured to perform these and other methods, and computer program products that include a computer readable storage medium storing instructions for execution to perform these and other methods are also provided.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

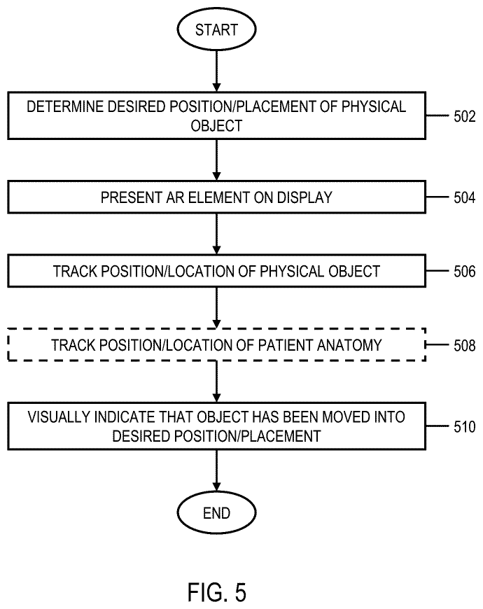

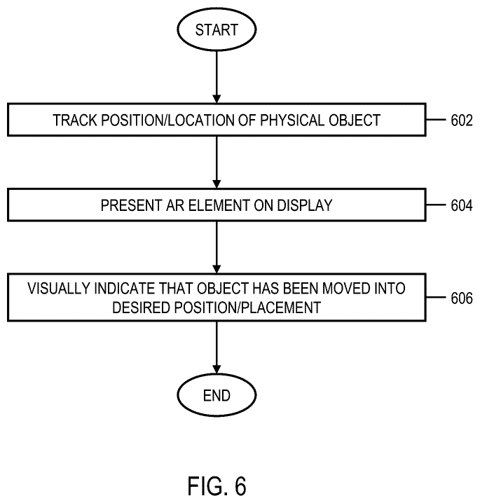

FIGS. 5 and 6 depict example processes for tracking, locating, and/or placing an object relative to patient anatomy during a surgical procedure, in accordance with aspects described herein. The process can be performed by a computer system executing software to perform aspects discussed herein, for instance a computer system as described above with reference to FIGS. 3 and 4, as an example.

Referring initially to FIG. 5, a process is presented that focuses on maintaining an augmented reality (AR) at a desired position of an object (e.g., implant) and visually indicating when the object has been correctly placed. The process includes determining (502) a desired position and placement for a physical object to be positioned and placed relative to a patient anatomy. In embodiments, the desired position and placement is dictated by a surgical plan for a patient. The physical object may be an implant, as an example.

The process continues by presenting (504), on a display device, an augmented reality (AR) element overlying a view of a surgical environment. The surgical environment includes the patient anatomy. The presenting of the AR element positions and places the AR element at the desired position and placement for the physical object, in order to facilitate proper placement of the physical object relative to the patient anatomy. In embodiments, the AR element is a digital model of the physical object.

In embodiments, the process obtains a video stream depicting the view of the surgical environment, including a depiction of the patient anatomy, and presents the video stream on the display device. The presenting of the AR element on the display device can augment the video stream to include the AR element at the desired position and placement for the physical object to be placed relative to the patient anatomy being depicted in the video stream. In examples, as part of presenting the video stream, and as the physical object is positioned relative to the patient anatomy, the process can zoom-in on an area of the surgical environment where the AR element is presented.

Additionally or alternatively, in embodiments the display device can include a transparent display through which a user sees the view of the surgical environment. The AR element can be presented on the transparent display and augment the user's view of the surgical environment through the transparent display. In embodiments, the transparent display is provided as part of a smart wearable glasses device.

Continuing with FIG. 5, the process tracks (506) position and location of the physical object as the physical object is moved in the surgical environment (e.g., moved to position and place the physical object relative to the patient anatomy). In embodiments, tracking the position and location of the physical object is performed using at least one of: one or more markers on the physical object, a shape matching algorithm that identifies position and location of the physical object in the view based on a known shape of the physical object, or tracking location of at least one other physical object that has a fixed, known location relative to the physical object.

Optionally the process includes tracking (508) location and position of the patient anatomy in the surgical environment. In these embodiments, based on the patient anatomy being repositioned, presenting the AR element repositions the AR element to maintain the AR element at the desired position and placement for the physical object to be placed relative to the patient anatomy.

The tracking 506 (and optionally 508) can be performed continuously/substantially continuously, with a check/determination as to whether the position and location of the physical object matches to the desired position and placement for the physical object. Based on the position and location of the physical object matching to the desired position and placement for the physical object, the process can visually indicate (510) on the display device that the physical object has been moved into the desired position and placement relative to the patient anatomy. In embodiments, the visually indicating includes modifying a visual presentation of the AR element on the display device. For example, the AR element may initially be at least partially a first color or pattern, and the visually indicating can include changing the first color or pattern to a different color or pattern.

In embodiments, the surgical plan can include one or more desired cuts to patient anatomy, and the process can determine that the one or more desired cuts have been made to the patient anatomy and further perform the determining 502 and the presenting 504, i.e., determining the desired position and placement for the physical object and presenting the AR element based on the one or more desired cuts having been made.

In some embodiments, the AR element can be a first AR element and the process can further include presenting, on the display device, a second AR element that overlays the physical object in the view of the surgical environment.

Referring now to FIG. 6, a process is presented that focuses on maintaining an AR element tracking/overlaying an object (e.g., an implant) as it is moved into position, and visually indicating correct placement when in position. The process includes tracking (602) position and location of a physical object in a surgical environment as the physical object is moved in the surgical environment. In embodiments, the physical object is an implant. In embodiments, tracking the position and location of the physical object is performed using at least one of: one or more markers on the physical object, a shape matching algorithm that identifies position and location of the physical object in the view based on a known shape of the physical object, or tracking location of at least one other physical object that has a fixed, known location relative to the physical object.

Continuing with FIG. 6, the process presents (604), on a display device, an augmented reality (AR) element that overlays the physical object in a view of the surgical environment. The view shows the physical object and a patient anatomy relative to which the physical object is to be positioned and placed in accordance with a desired position and placement for the physical object as dictated by a surgical plan for a patient. The presenting can maintain, based on the tracking, the AR element overlaying the physical object in the view as the physical object is moved in the surgical environment. In embodiments, the AR element is a digital model of the physical object.

In embodiments, the process obtains a video stream depicting the view of the surgical environment, including a depiction of the patient anatomy, and presents the video stream on the display device. The presenting the AR element on the display device can augment the video stream to include the AR element overlaying the physical object in the view of the surgical environment. In examples, as part of presenting the video stream, and as the physical object is positioned relative to the patient anatomy, the process can zoom-in on an area of the surgical environment showing a position of the desired position and placement for the physical object.

Additionally or alternatively, in embodiments the display device can include a transparent display through which a user sees the view of the surgical environment. The AR element can be presented on the transparent display and augment the user's view of the surgical environment through the transparent display. In embodiments, the transparent display is provided as part of a smart wearable glasses device.

The tracking 602 and presenting 604 can be performed continuously/substantially continuously, with a check/determination as to whether the position and location of the physical object matches to the desired position and placement for the physical object. Based on the position and location of the physical object matching to the desired position and placement for the physical object, the process can visually indicate (606) on the display device that the physical object has been moved into the desired position and placement relative to the patient anatomy. In embodiments, the visually indicating includes modifying a visual presentation of the AR element on the display device. For example, the AR element may initially be at least partially a first color or pattern, and the visually indicating can include changing the first color or pattern to a different color or pattern.

In some embodiments, the AR element can be a first AR element and the process can further include presenting, on the display device, a second AR element positioned and placed relative to the patient anatomy to indicate the desired position and placement for the physical object.

Although various embodiments are described above, these are only examples.

Provided is a small sampling of embodiments of the present disclosure, as described herein:

A1. A computer-implemented method comprising: determining a desired position and placement for a physical object to be positioned and placed relative to a patient anatomy, the desired position and placement being dictated by a surgical plan for a patient; presenting, on a display device, an augmented reality (AR) element overlying a view of a surgical environment, wherein the surgical environment includes the patient anatomy, and wherein the presenting positions and places the AR element at the desired position and placement for the physical object to facilitate proper placement of the physical object relative to the patient anatomy; tracking position and location of the physical object as the physical object is moved in the surgical environment to position and place the physical object relative to the patient anatomy; and based on the position and location of the physical object matching to the desired position and placement for the physical object, visually indicating on the display device that the physical object has been moved into the desired position and placement relative to the patient anatomy.

A2. The method of A1, further comprising: obtaining a video stream depicting the view of the surgical environment, including a depiction of the patient anatomy; and presenting the video stream on the display device, wherein the presenting the AR element on the display device augments the video stream to include the AR element at the desired position and placement for the physical object to be placed relative to the patient anatomy as depicted in the video stream.

A3. The method of A2, further comprising, as part of presenting the video stream, and as the physical object is positioned relative to the patient anatomy, zooming-in on an area of the surgical environment where the AR element is presented.

A4. The method of A1, wherein the display device comprises a transparent display through which a user sees the view of the surgical environment, wherein the AR element is presented on the transparent display and augments the user's view of the surgical environment through the transparent display.

A5. The method of A4, wherein the transparent display is provided as part of a smart wearable glasses device.

A6. The method of A1, A2, A3, A4, or A5, wherein the visually indicating comprises modifying a visual presentation of the AR element on the display device.

A7. The method of A6, wherein the AR element is initially at least partially a first color or pattern and wherein the visually indicating comprising changing the first color or pattern to a different color or pattern.

A8. The method of A1, A2, A3, A4, or A5, wherein the tracking the position and location of the physical object is performed using at least one of: one or more markers on the physical object; a shape matching algorithm that identifies position and location of the physical object in the view based on a known shape of the physical object; or tracking location of at least one other physical object that has a fixed, known location relative to the physical object.

A9. The method of A1, A2, A3, A4, or A5, further comprising tracking location and position of the patient anatomy in the surgical environment, wherein, based on the patient anatomy being repositioned, the presenting the AR element repositions the AR element to maintain the AR element at the desired position and placement for the physical object to be placed relative to the patient anatomy.

A10. The method of A1, A2, A3, A4, or A5, wherein the surgical plan comprises one or more desired cuts to patient anatomy, wherein the method determines that the one or more desired cuts have been made to the patient anatomy and further performs the determining the desired position and placement for the physical object and the presenting the AR element based on the one or more desired cuts having been made.

A11. The method of A1, A2, A3, A4, or A5, wherein the AR element is a first AR element and wherein the method further comprises presenting, on the display device, a second AR element that overlays the physical object in the view of the surgical environment.

A12. The method of A1, A2, A3, A4, or A5, wherein the physical object is an implant.

A13. The method of A1, A2, A3, A4, or A5, wherein the AR element is a digital model of the physical object.

A14. A computer system comprising: a memory; and a processor in communication with the memory, wherein the computer system is configured to perform a method comprising: determining a desired position and placement for a physical object to be positioned and placed relative to a patient anatomy, the desired position and placement being dictated by a surgical plan for a patient; presenting, on a display device, an augmented reality (AR) element overlying a view of a surgical environment, wherein the surgical environment includes the patient anatomy, and wherein the presenting positions and places the AR element at the desired position and placement for the physical object to facilitate proper placement of the physical object relative to the patient anatomy; tracking position and location of the physical object as the physical object is moved in the surgical environment to position and place the physical object relative to the patient anatomy; and based on the position and location of the physical object matching to the desired position and placement for the physical object, visually indicating on the display device that the physical object has been moved into the desired position and placement relative to the patient anatomy.

A15. The computer system of A14, further comprising: obtaining a video stream depicting the view of the surgical environment, including a depiction of the patient anatomy; and presenting the video stream on the display device, wherein the presenting the AR element on the display device augments the video stream to include the AR element at the desired position and placement for the physical object to be placed relative to the patient anatomy as depicted in the video stream.

A16. The computer system of A15, further comprising, as part of presenting the video stream, and as the physical object is positioned relative to the patient anatomy, zooming-in on an area of the surgical environment where the AR element is presented.

A17. The computer system of A14, wherein the display device comprises a transparent display through which a user sees the view of the surgical environment, wherein the AR element is presented on the transparent display and augments the user's view of the surgical environment through the transparent display.

A18. The computer system of A17, wherein the transparent display is provided as part of a smart wearable glasses device.

A19. The computer system of A14, A15, A16, A17, or A18, wherein the visually indicating comprises modifying a visual presentation of the AR element on the display device.

A20. The computer system of A19, wherein the AR element is initially at least partially a first color or pattern and wherein the visually indicating comprising changing the first color or pattern to a different color or pattern.

A21. The computer system of A14, A15, A16, A17, or A18, wherein the tracking the position and location of the physical object is performed using at least one of: one or more markers on the physical object; a shape matching algorithm that identifies position and location of the physical object in the view based on a known shape of the physical object; or tracking location of at least one other physical object that has a fixed, known location relative to the physical object.

A22. The computer system of A14, A15, A16, A17, or A18, further comprising tracking location and position of the patient anatomy in the surgical environment, wherein, based on the patient anatomy being repositioned, the presenting the AR element repositions the AR element to maintain the AR element at the desired position and placement for the physical object to be placed relative to the patient anatomy.

A23. The computer system of A14, A15, A16, A17, or A18, wherein the surgical plan comprises one or more desired cuts to patient anatomy, wherein the method determines that the one or more desired cuts have been made to the patient anatomy and further performs the determining the desired position and placement for the physical object and the presenting the AR element based on the one or more desired cuts having been made.

A24. The computer system of A14, A15, A16, A17, or A18, wherein the AR element is a first AR element and wherein the method further comprises presenting, on the display device, a second AR element that overlays the physical object in the view of the surgical environment.

A25. The computer system of A14, A15, A16, A17, or A18, wherein the physical object is an implant.

A26. The computer system of A14, A15, A16, A17, or A18, wherein the AR element is a digital model of the physical object.

A27. A computer program product comprising: a computer readable storage medium readable by a processing circuit and storing instructions for execution by the processing circuit for performing a method comprising: determining a desired position and placement for a physical object to be positioned and placed relative to a patient anatomy, the desired position and placement being dictated by a surgical plan for a patient; presenting, on a display device, an augmented reality (AR) element overlying a view of a surgical environment, wherein the surgical environment includes the patient anatomy, and wherein the presenting positions and places the AR element at the desired position and placement for the physical object to facilitate proper placement of the physical object relative to the patient anatomy; tracking position and location of the physical object as the physical object is moved in the surgical environment to position and place the physical object relative to the patient anatomy; and based on the position and location of the physical object matching to the desired position and placement for the physical object, visually indicating on the display device that the physical object has been moved into the desired position and placement relative to the patient anatomy.

A28. The computer program product of A27, further comprising: obtaining a video stream depicting the view of the surgical environment, including a depiction of the patient anatomy; and presenting the video stream on the display device, wherein the presenting the AR element on the display device augments the video stream to include the AR element at the desired position and placement for the physical object to be placed relative to the patient anatomy as depicted in the video stream.

A29. The computer program product of A28, further comprising, as part of presenting the video stream, and as the physical object is positioned relative to the patient anatomy, zooming-in on an area of the surgical environment where the AR element is presented.

A30. The computer program product of A27, wherein the display device comprises a transparent display through which a user sees the view of the surgical environment, wherein the AR element is presented on the transparent display and augments the user's view of the surgical environment through the transparent display.

A31. The computer program product of A30, wherein the transparent display is provided as part of a smart wearable glasses device.

A32. The computer program product of A27, A28, A29, A30, or A31, wherein the visually indicating comprises modifying a visual presentation of the AR element on the display device.

A33. The computer program product of A32, wherein the AR element is initially at least partially a first color or pattern and wherein the visually indicating comprising changing the first color or pattern to a different color or pattern.

A34. The computer program product of A27, A28, A29, A30, or A31, wherein the tracking the position and location of the physical object is performed using at least one of: one or more markers on the physical object; a shape matching algorithm that identifies position and location of the physical object in the view based on a known shape of the physical object; or tracking location of at least one other physical object that has a fixed, known location relative to the physical object.

A35. The computer program product of A27, A28, A29, A30, or A31, further comprising tracking location and position of the patient anatomy in the surgical environment, wherein, based on the patient anatomy being repositioned, the presenting the AR element repositions the AR element to maintain the AR element at the desired position and placement for the physical object to be placed relative to the patient anatomy.

A36. The computer program product of A27, A28, A29, A30, or A31, wherein the surgical plan comprises one or more desired cuts to patient anatomy, wherein the method determines that the one or more desired cuts have been made to the patient anatomy and further performs the determining the desired position and placement for the physical object and the presenting the AR element based on the one or more desired cuts having been made.

A37. The computer program product of A27, A28, A29, A30, or A31, wherein the AR element is a first AR element and wherein the method further comprises presenting, on the display device, a second AR element that overlays the physical object in the view of the surgical environment.

A38. The computer program product of A27, A28, A29, A30, or A31, wherein the physical object is an implant.

A39. The computer program product of A27, A28, A29, A30, or A31, wherein the AR element is a digital model of the physical object.

B1. A computer-implemented method comprising: tracking position and location of a physical object in a surgical environment as the physical object is moved in the surgical environment; presenting, on a display device, an augmented reality (AR) element that overlays the physical object in a view of the surgical environment, the view showing the physical object and a patient anatomy relative to which the physical object is to be positioned and placed in accordance with a desired position and placement for the physical object as dictated by a surgical plan for a patient, wherein the presenting maintains, based on the tracking, the AR element overlaying the physical object in the view as the physical object is moved in the surgical environment; and based on the position and location of the physical object matching to the desired position and placement for the physical object, visually indicating on the display device that the physical object has been moved into the desired position and placement relative to the patient anatomy.

B2. The method of B1, further comprising: obtaining a video stream depicting the view of the surgical environment, including a depiction of the patient anatomy; and presenting the video stream on the display device, wherein the presenting the AR element on the display device augments the video stream to include the AR element overlaying the physical object in the view of the surgical environment.

B3. The method of B2, further comprising, as part of presenting the video stream, and as the physical object is positioned relative to the patient anatomy, zooming-in on an area of the surgical environment showing a position of the desired position and placement for the physical object.

B4. The method of B1, wherein the display device comprises a transparent display through which a user sees the view of the surgical environment, wherein the AR element is presented on the transparent display and augments the user's view of the surgical environment through the transparent display.

B5. The method of B4, wherein the transparent display is provided as part of a smart wearable glasses device.

B6. The method of B1, B2, B3, B4, or B5, wherein the visually indicating comprises modifying a visual presentation of the AR element on the display device.

B7. The method of B6, wherein the AR element is initially at least partially a first color or pattern and wherein the visually indicating comprising changing the first color or pattern to a different color or pattern.

B8. The method of B1, B2, B3, B4, or B5, wherein the tracking the position and location of the physical object is performed using at least one of: one or more markers on the physical object; a shape matching algorithm that identifies position and location of the physical object in the view based on a known shape of the physical object; or tracking location of at least one other physical object that has a fixed, known location relative to the physical object.

B9. The method of B1, B2, B3, B4, or B5, wherein the AR element is a first AR element and wherein the method further comprises presenting, on the display device, a second AR element positioned and placed relative to the patient anatomy to indicate the desired position and placement for the physical object.

B10. The method of B1, B2, B3, B4, or B5, wherein the AR element is a digital model of the physical object.

B11. The method of B1, B2, B3, B4, or B5, wherein the physical object is an implant.

B12. A computer system comprising: a memory; and a processor in communication with the memory, wherein the computer system is configured to perform a method comprising: tracking position and location of a physical object in a surgical environment as the physical object is moved in the surgical environment; presenting, on a display device, an augmented reality (AR) element that overlays the physical object in a view of the surgical environment, the view showing the physical object and a patient anatomy relative to which the physical object is to be positioned and placed in accordance with a desired position and placement for the physical object as dictated by a surgical plan for a patient, wherein the presenting maintains, based on the tracking, the AR element overlaying the physical object in the view as the physical object is moved in the surgical environment; and based on the position and location of the physical object matching to the desired position and placement for the physical object, visually indicating on the display device that the physical object has been moved into the desired position and placement relative to the patient anatomy.

B13. The computer system of B12, wherein the method further comprises: obtaining a video stream depicting the view of the surgical environment, including a depiction of the patient anatomy; and presenting the video stream on the display device, wherein the presenting the AR element on the display device augments the video stream to include the AR element overlaying the physical object in the view of the surgical environment.

B14. The computer system of B13, wherein the method further comprises, as part of presenting the video stream, and as the physical object is positioned relative to the patient anatomy, zooming-in on an area of the surgical environment showing a position of the desired position and placement for the physical object.

B15. The computer system of B12, wherein the display device comprises a transparent display through which a user sees the view of the surgical environment, wherein the AR element is presented on the transparent display and augments the user's view of the surgical environment through the transparent display.

B16. The computer system of B15, wherein the transparent display is provided as part of a smart wearable glasses device.

B17. The computer system of B12, B13, B14, B15, or B16, wherein the visually indicating comprises modifying a visual presentation of the AR element on the display device.

B18. The computer system of B17, wherein the AR element is initially at least partially a first color or pattern and wherein the visually indicating comprising changing the first color or pattern to a different color or pattern.

B19. The computer system of B12, B13, B14, B15, or B16, wherein the tracking the position and location of the physical object is performed using at least one of: one or more markers on the physical object; a shape matching algorithm that identifies position and location of the physical object in the view based on a known shape of the physical object; or tracking location of at least one other physical object that has a fixed, known location relative to the physical object.

B20. The computer system of B12, B13, B14, B15, or B16, wherein the AR element is a first AR element and wherein the method further comprises presenting, on the display device, a second AR element positioned and placed relative to the patient anatomy to indicate the desired position and placement for the physical object.

B21. The computer system of B12, B13, B14, B15, or B16, wherein the AR element is a digital model of the physical object.

B22. The computer system of B12, B13, B14, B15, or B16, wherein the physical object is an implant.

B23. A computer program product comprising: a computer readable storage medium readable by a processing circuit and storing instructions for execution by the processing circuit for performing a method comprising: tracking position and location of a physical object in a surgical environment as the physical object is moved in the surgical environment; presenting, on a display device, an augmented reality (AR) element that overlays the physical object in a view of the surgical environment, the view showing the physical object and a patient anatomy relative to which the physical object is to be positioned and placed in accordance with a desired position and placement for the physical object as dictated by a surgical plan for a patient, wherein the presenting maintains, based on the tracking, the AR element overlaying the physical object in the view as the physical object is moved in the surgical environment; and based on the position and location of the physical object matching to the desired position and placement for the physical object, visually indicating on the display device that the physical object has been moved into the desired position and placement relative to the patient anatomy.

B24. The computer program product of B23, wherein the method further comprises: obtaining a video stream depicting the view of the surgical environment, including a depiction of the patient anatomy; and presenting the video stream on the display device, wherein the presenting the AR element on the display device augments the video stream to include the AR element overlaying the physical object in the view of the surgical environment.

B25. The computer program product of B24, wherein the method further comprises, as part of presenting the video stream, and as the physical object is positioned relative to the patient anatomy, zooming-in on an area of the surgical environment showing a position of the desired position and placement for the physical object.

B26. The computer program product of B23, wherein the display device comprises a transparent display through which a user sees the view of the surgical environment, wherein the AR element is presented on the transparent display and augments the user's view of the surgical environment through the transparent display.

B27. The computer program product of B26, wherein the transparent display is provided as part of a smart wearable glasses device.

B28. The computer program product of B23, B24, B25, B26, or B27, wherein the visually indicating comprises modifying a visual presentation of the AR element on the display device.

B29. The computer program product of B28, wherein the AR element is initially at least partially a first color or pattern and wherein the visually indicating comprising changing the first color or pattern to a different color or pattern.

B30. The computer program product of B23, B24, B25, B26, or B27, wherein the tracking the position and location of the physical object is performed using at least one of: one or more markers on the physical object; a shape matching algorithm that identifies position and location of the physical object in the view based on a known shape of the physical object; or tracking location of at least one other physical object that has a fixed, known location relative to the physical object.

B31. The computer program product of B23, B24, B25, B26, or B27, wherein the AR element is a first AR element and wherein the method further comprises presenting, on the display device, a second AR element positioned and placed relative to the patient anatomy to indicate the desired position and placement for the physical object.

B32. The computer program product of B23, B24, B25, B26, or B27, wherein the AR element is a digital model of the physical object.

B33. The computer program product of B23, B24, B25, B26, or B27, wherein

the physical object is an implant.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
determining a desired position and placement for a physical object to be positioned and placed relative to a patient anatomy, the desired position and placement being dictated by a surgical plan for a patient;
presenting, on a display device, an augmented reality (AR) element overlying a view of a surgical environment, wherein the surgical environment includes the patient anatomy, and wherein the presenting positions and places the AR element at the desired position and placement for the physical object to facilitate proper placement of the physical object relative to the patient anatomy;
tracking position and location of the physical object as the physical object is moved in the surgical environment to position and place the physical object relative to the patient anatomy; and
based on the position and location of the physical object matching to the desired position and placement for the physical object, visually indicating on the display device that the physical object has been moved into the desired position and placement relative to the patient anatomy.

2. The method of claim 1, further comprising:
obtaining a video stream depicting the view of the surgical environment, including a depiction of the patient anatomy; and
presenting the video stream on the display device, wherein the presenting the AR element on the display device augments the video stream to include the AR element at the desired position and placement for the physical object to be placed relative to the patient anatomy as depicted in the video stream.

3. The method of claim 2, further comprising, as part of presenting the video stream, and as the physical object is positioned relative to the patient anatomy, zooming-in on an area of the surgical environment where the AR element is presented.

4. The method of claim 1, wherein the display device comprises a transparent display through which a user sees the view of the surgical environment, wherein the AR element is presented on the transparent display and augments the user's view of the surgical environment through the transparent display.

5. The method of claim 4, wherein the transparent display is provided as part of a smart wearable glasses device.

6. The method of claim 1, wherein the visually indicating comprises modifying a visual presentation of the AR element on the display device.

7. The method of claim 6, wherein the AR element is initially at least partially a first color or pattern and wherein the visually indicating comprises changing the first color or pattern to a different color or pattern.

8. The method of claim 1, wherein the tracking the position and location of the physical object is performed using at least one of:
one or more markers on the physical object;
a shape matching algorithm that identifies position and location of the physical object in the view based on a known shape of the physical object; or
tracking location of at least one other physical object that has a fixed, known location relative to the physical object.

9. The method of claim 1, further comprising tracking location and position of the patient anatomy in the surgical environment, wherein, based on the patient anatomy being repositioned, the presenting the AR element repositions the AR element to maintain the AR element at the desired position and placement for the physical object to be placed relative to the patient anatomy.

10. The method of claim 1, wherein the surgical plan comprises one or more desired cuts to patient anatomy, wherein the method determines that the one or more desired cuts have been made to the patient anatomy and further performs the determining the desired position and placement for the physical object and the presenting the AR element based on the one or more desired cuts having been made.

11. The method of claim 1, wherein the AR element is a first AR element and wherein the method further comprises presenting, on the display device, a second AR element that overlays the physical object in the view of the surgical environment.

12. The method of claim 1, wherein the physical object is an implant.

13. The method of claim 1, wherein the AR element is a digital model of the physical object.

14. A computer system comprising:
a memory; and
a processor in communication with the memory, wherein the computer system is configured to perform a method comprising:
determining a desired position and placement for a physical object to be positioned and placed relative to a patient anatomy, the desired position and placement being dictated by a surgical plan for a patient;
presenting, on a display device, an augmented reality (AR) element overlying a view of a surgical environment, wherein the surgical environment includes the patient anatomy, and wherein the presenting positions and places the AR element at the desired position and placement for the physical object to facilitate proper placement of the physical object relative to the patient anatomy;
tracking position and location of the physical object as the physical object is moved in the surgical environment to position and place the physical object relative to the patient anatomy; and
based on the position and location of the physical object matching to the desired position and placement for the physical object, visually indicating on the display device that the physical object has been moved into the desired position and placement relative to the patient anatomy.

15. The computer system of claim 14, wherein the physical object is an implant, and wherein the method further comprises:
obtaining a video stream depicting the view of the surgical environment, including a depiction of the patient anatomy; and
presenting the video stream on the display device, wherein the presenting the AR element on the display device augments the video stream to include the AR element at the desired position and placement for the physical object to be placed relative to the patient anatomy as depicted in the video stream.

16. A computer-implemented method comprising:
tracking position and location of a physical object in a surgical environment as the physical object is moved in the surgical environment;
presenting, on a display device, an augmented reality (AR) element that overlays the physical object in a view of the surgical environment, the view showing the physical object and a patient anatomy relative to which the physical object is to be positioned and placed in accordance with a desired position and placement for the physical object as dictated by a surgical plan for a patient, wherein the presenting maintains, based on the tracking, the AR element overlaying the physical object in the view as the physical object is moved in the surgical environment; and
based on the position and location of the physical object matching to the desired position and placement for the physical object, visually indicating on the display device that the physical object has been moved into the desired position and placement relative to the patient anatomy.

17. The method of claim 16, further comprising:
obtaining a video stream depicting the view of the surgical environment, including a depiction of the patient anatomy; and
presenting the video stream on the display device, wherein the presenting the AR element on the display device augments the video stream to include the AR element overlaying the physical object in the view of the surgical environment.

18. The method of claim 17, further comprising, as part of presenting the video stream, and as the physical object is positioned relative to the patient anatomy, zooming-in on an area of the surgical environment showing a position of the desired position and placement for the physical object.

19. The method of claim 16, wherein the display device comprises a transparent display through which a user sees the view of the surgical environment, wherein the AR element is presented on the transparent display and augments the user's view of the surgical environment through the transparent display.

20. The method of claim 19, wherein the transparent display is provided as part of a smart wearable glasses device.

21. The method of claim 16, wherein the visually indicating comprises modifying a visual presentation of the AR element on the display device.

22. The method of claim 21, wherein the AR element is initially at least partially a first color or pattern and wherein the visually indicating comprises changing the first color or pattern to a different color or pattern.

23. The method of claim 16, wherein the tracking the position and location of the physical object is performed using at least one of:
one or more markers on the physical object;
a shape matching algorithm that identifies position and location of the physical object in the view based on a known shape of the physical object; or
tracking location of at least one other physical object that has a fixed, known location relative to the physical object.

24. The method of claim 16, wherein the AR element is a first AR element and wherein the method further comprises presenting, on the display device, a second AR element positioned and placed relative to the patient anatomy to indicate the desired position and placement for the physical object.

25. The method of claim 16, wherein the AR element is a digital model of the physical object.

26. The method of claim 16, wherein the physical object is an implant.

27. A computer system comprising:
a memory; and
a processor in communication with the memory, wherein the computer system is configured to perform a method comprising:
tracking position and location of a physical object in a surgical environment as the physical object is moved in the surgical environment;
presenting, on a display device, an augmented reality (AR) element that overlays the physical object in a view of the surgical environment, the view showing the physical object and a patient anatomy relative to which the physical object is to be positioned and placed in accordance with a desired position and placement for the physical object as dictated by a surgical plan for a patient, wherein the presenting maintains, based on the tracking, the AR element overlaying the physical object in the view as the physical object is moved in the surgical environment; and
based on the position and location of the physical object matching to the desired position and placement for the physical object, visually indicating on the display device that the physical object has been moved into the desired position and placement relative to the patient anatomy.

28. The computer system of claim 27, wherein the physical object is an implant, wherein the visually indicating comprises modifying a visual presentation of the AR element on the display device, and wherein the method further comprises:
obtaining a video stream depicting the view of the surgical environment, including a depiction of the patient anatomy; and
presenting the video stream on the display device, wherein the presenting the AR element on the display device augments the video stream to include the AR element overlaying the physical object in the view of the surgical environment.
